(19) United States
(12) Patent Application Publication
(10) Pub. No.: US 2025/0213886 A1
(43) Pub. Date: Jul. 3, 2025

(54) ENERGY PATCH

(71) Applicant: ENERGREEN HEALTH INDUSTRY CHAIN CO. LTD., New Taipei City (TW)

(72) Inventor: Chin Cho HUNG, Taichung City (TW)

(21) Appl. No.: 18/848,554

(22) PCT Filed: Mar. 20, 2023
(86) PCT No.: PCT/CN2023/082545

§ 371 Date: Sep. 19, 2024

(30) Foreign Application Priority Data
Mar. 21, 2022 (CN) 202210275315.1

(51) Int. Cl. A61N 5/06; A61K 36/28

(52) U.S. Cl. CPC A61N 5/0625; A61K 36/28; A61N 2005/0645; A61N 2005/066

(57) ABSTRACT

An energy patch includes a body, a dressing, and an energy-transmitting material. The body is adapted to be repeatedly attached to a skin of a human body. The dressing is disposed in the body. The energy-transmitting material is disposed in the body and the energy-transmitting material is adapted to transmit energy generated by the dressing.

### FIELD OF THE INVENTION

The present invention relates to a patch, and more particularly to an energy patch.

### BACKGROUND OF THE INVENTION

The work styles of most modern people require them to use computers for long periods of time, so they are prone to problems such as stiffness and soreness in the shoulders and necks. Also, people have paid more attention to physical health and often take time to exercise after getting off work in recent years; however, because most people are accustomed to a sedentary lifestyle for long periods of time, they are more likely to suffer from muscle soreness.

Generally, muscle soreness can be relieved by rest or massage, and using pain relief patches can relieve discomfort more quickly. However, most of the conventional pain relief patches are only allow for using once, resulting in a large amount of material waste.

### SUMMARY OF THE INVENTION

The present invention provides an energy patch to have an advantage of being able to be reusable.

The energy patch provided by the invention includes a body, a dressing, and an energy-transmitting material. The body is adapted to be repeatedly attached to a skin of human body. The dressing is disposed in the body. The energy-transmitting material is disposed in the body and the energy-transmitting material is adapted to transmit energy generated by the dressing.

In an embodiment of the invention, a material of the body can includes silicone, rubber, plastic, polyester, artificial fiber, acrylic, rayon, acetate, elastane, lycra, nylon, latex, polyether polyurethane, foam, liquid gel, glass, stainless steel, steel or iron.

In an embodiment of the invention, the energy-transmitting material includes, for example, graphene.

In an embodiment of the invention, the dressing includes, for example, saussurea involucrata and a far-infrared radiation material.

In an embodiment of the invention, the dressing and the energy-transmitting material can be in a powdered form and are evenly distributed in the body.

In an embodiment of the invention, the body can have a surface and an attachment surface opposite to the surface, and the surface has a plurality of convex portions.

In an embodiment of the invention, the body has, for example, a plurality of air permeability holes.

In an embodiment of the invention, hole diameters of the air permeability holes are, for example, between 0.1-1 mm.

In an embodiment of the invention, the air permeability holes are distributed evenly.

In an embodiment of the invention, the body has an attachment surface and two side edges, the two side edges are respectively located on two opposite sides of the attachment surface, and the two side edges each have a concave portion recessed toward each other.

The energy patch of the invention uses the body able to be repeatedly attached to a skin, and the dressing and the energy-transmitting material are disposed in the body. Thus, even after the energy patch has been used many times, the dressing and the energy-transmitting material still can be remained in the body to continuously provide the effect of relieving discomfort of an affected area. Based on the above, the energy patch of the invention has the advantage of being reusable, thereby avoiding material waste.

The above description is only an overview of the art of the present invention, in order to have a clearer understanding of the technical means of the present invention, which can be implemented according to the contents of the description, and in order to make the above and other objects, features and advantages of the present invention more obvious and understandable, and the preferred embodiments are specifically cited below and described in detail with reference to the accompanying drawings.

### BRIEF DESCRIPTION OF THE DRAWINGS

FIG. 1 is a schematic diagram of an energy patch in an embodiment of the invention.

FIG. 2 is a schematic diagram of an attachment surface of the energy patch in FIG. 1.

FIG. 3 is a schematic top view of the energy patch in FIG. 1.

FIG. 4 is a schematic side view of the energy patch in FIG. 1.

### DETAILED DESCRIPTION OF PREFERRED EMBODIMENTS

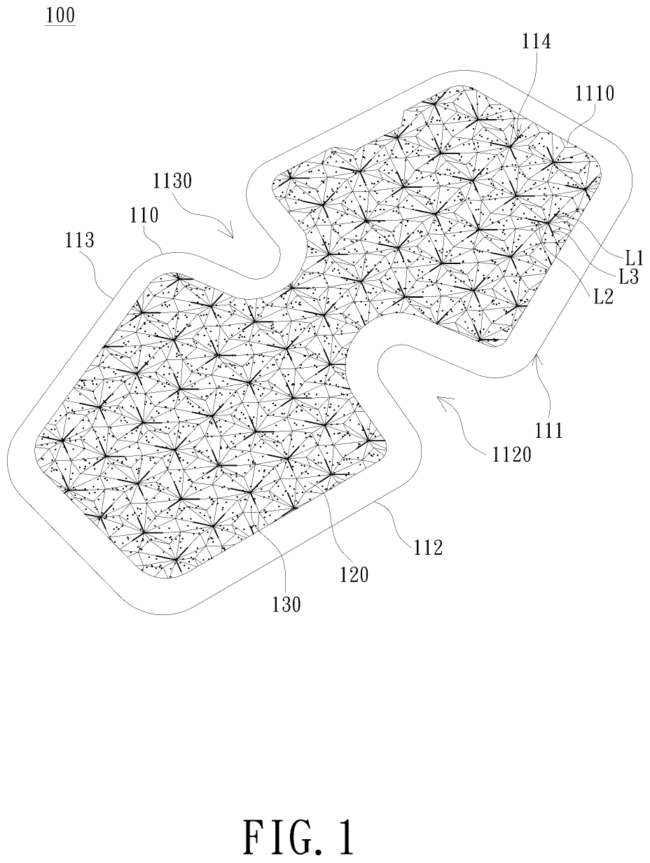

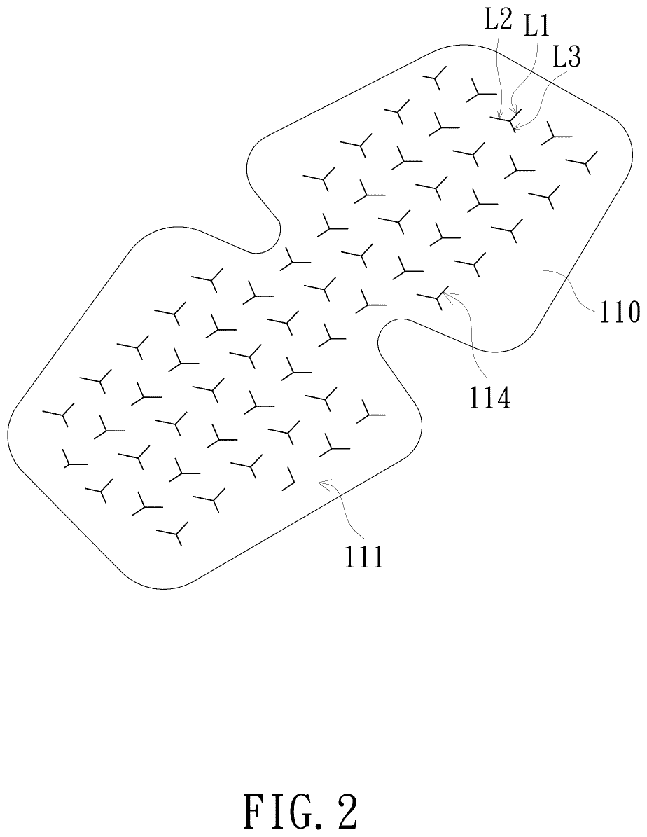

FIG. 1 is a schematic diagram of an energy patch in an embodiment of the invention. FIG. 2 is a schematic diagram of an attachment surface of the energy patch in FIG. 1. Referring to FIGS. 1 and 2, an energy patch 100 includes a body 110, a dressing 120, and an energy-transmitting material 130. The body 110 is adapted to be attached to a skin of a human body. Specifically, the body 110 itself is able to be attached to the skin of the human body without requiring additional adhesive materials for adhering to the skin, but other embodiments are not limited thereto. It should be noted that the body 110 is, for example, attached to the skin with an attachment surface 111 in FIG. 2 in this embodiment. The dressing 120 is disposed in the body 110. The energy-transmitting material 130 is disposed in the body 110 and the energy-transmitting material is adapted to transmit energy generated by the dressing 120. The aforementioned energy in this embodiment is exemplified by thermal energy to provide a function of relieving muscle soreness in an affected area. It can be understood that the aforementioned energy is not limited to thermal energy in other embodiments, and the specific types of the energy is not limited by the invention.

The dressing 120 in this embodiment includes, for example, saussurea involucrate and a far-infrared radiation material to generate thermal energy to relieve the muscle soreness in the affected area. In an embodiment, the dressing 120 may further include carthamus tinctorius, lycopodii herba, hibiscus mutabilis, chuanxiong rhizoma, and cinnamomi ramulus, etc. It can be understood that the energy generated by the dressing 120 in this embodiment is not limited to thermal energy. For example, the dressing 120 can be used to reduce the temperature of the affected area to provide an effect similar to ice pack in other embodiments.

The energy-transmitting material 130 in this embodiment includes, for example, graphene to transmit the energy generated by the dressing 120 to the affected area. For example, the energy-transmitting material 130 is able to transmit the aforementioned thermal energy in this embodiment. However, the energy-transmitting material 130 is able to further transmit other types of energy in another embodiment. Additionally, the dressing 120 and the energy-transmitting material 130 in this embodiment can be in a powdered form and are evenly distributed in the body 110 to generate and transmit the thermal energy evenly to the affected area

A material of the body 110 may include silicone so the body 110 itself is able to be repeatedly attached to the skin to provide a reusable function. However, an adhesive material (not shown in figures) may be provided on the attachment surface 111 in an embodiment so that the body 110 can be attached more firmly to the skin. Specifically, the aforementioned adhesive material may be mixed with the dressing 120 so that the energy generated by the dressing 120 can be more quickly transmitted to the affected area to relieve the discomfort of the affected area more effectively. Incidentally, the aforementioned adhesive material includes, for example, a glue material, but the invention is not limited thereto. The material of the body 110 may include rubber or plastic in another embodiment, but the invention is not limited thereto. For example, in other embodiments, the material of the body 110 may include polyester, artificial fiber, acrylic, rayon, acetate, elastane, lycra, nylon, latex, polyether polyurethane, foam, liquid gel, glass, stainless steel, steel, or iron, etc.

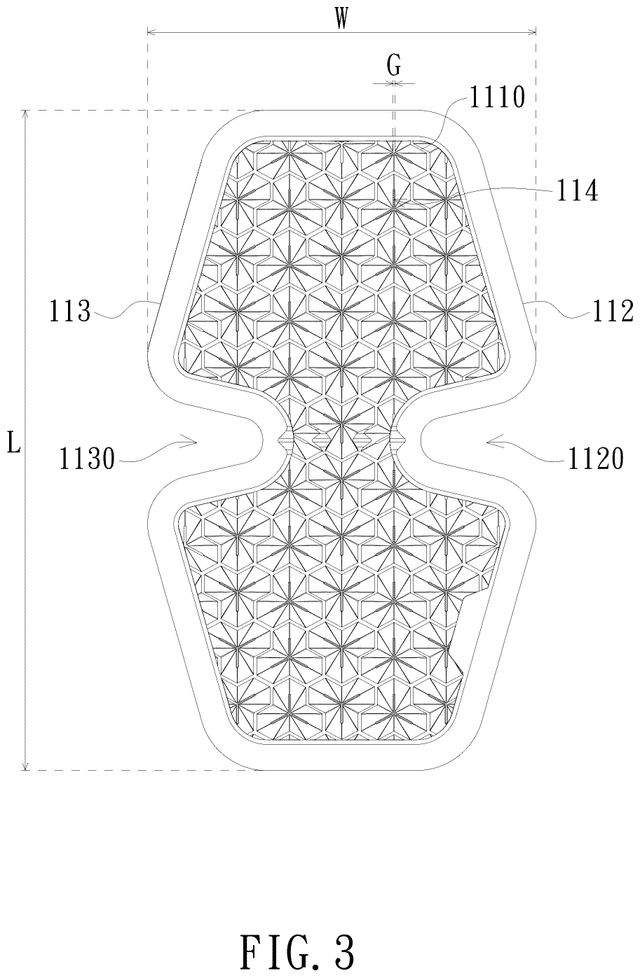

Referring to FIGS. 1 and 3 together, in this embodiment, the body 110 can have a surface (i.e., the surface shown in FIG. 3) opposite to the attachment surface 111, and the surface can have a plurality of convex portions 1110. Particularly, because the dressing 120 and the energy-transmitting material 130 are evenly distributed in the body 110, the number of the dressing 120 and the number of the energy-transmitting material 130 in the convex portions 1110 can be greater than those in other parts of the body 110. Thus, the convex portions 1110 are able to generate and transmit more thermal energy to the affected area to enhance the effect of hot compress of a part of the affected area and improve blood circulation of the part of the affected area. Based on the above, the convex portions 1110 are able to provide an effect similar to a localized massage on the muscles to more effectively relieve the discomfort of the affected area.

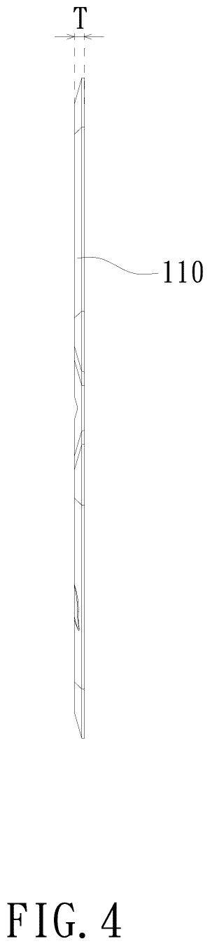

Referring to FIGS. 1 and 3 again, the body 110 in this embodiment further has, for example, side edges 112 and 113. The side edges 112 and 113 are respectively located on two opposite sides of the attachment surface 111, and the side edges 112 and 113 can each have concave portions 1120 and 1130 recessed toward each other. Specifically, the side edge 112 may have the concave portion 1120 depressed toward the side edge 113, and the side edge 113 may have the concave portion 1130 depressed toward the side edge 112. Therefore, the shape of the energy patch 100 can preferably fit the joint of the human body, thereby preventing the energy patch 100 from falling off due to the joint movement. Incidentally, as shown in FIG. 3, the maximum width W of the body 110 may be approximately 90-95 mm, the length of the concave portion 1120 depressed from the side edge 112 may be approximately 25-30 mm, and the length of another concave portion 1130 recessed from the side edge 113 is similar to that of the concave portion 1120. Also, the maximum length L of the body 110 may be between 155-165 mm; additionally, as shown in FIG. 4, the thickness T of the body 110 may be approximately between 2-3 mm, but these detailed features are not limited by the invention.

Referring to FIGS. 1, 2 and 3 together, the body 110 in this embodiment has, for example, a plurality of air permeability holes 114 to enhance the comfort of the affected area. Furthermore, each of the air permeability holes 114 is, for example distributed evenly so that the body 110 can provide a more even ventilation effect to further enhance the comfort of the affected area. However, the air permeability holes 114 may be distributed in localized areas of the body 110 in other embodiments. Additionally, as shown in FIG. 3, the hole diameters G of the air permeability holes 114 in this embodiment are, for example, between 0.1-1 mm such as about 2 mm, but other embodiments are not limited thereto. Incidentally, as shown in FIGS. 1 and 2, the shape of the air permeability hole 114 is illustrated by three intersecting slits L1, L2, and L3 in this embodiment, wherein the angle between any two adjacent slits L1, L2, and L3 may be about 120 degrees, and the three slits L1, L2, and L3 may each be located on the convex portions 1110. It can be understood that the shape of the air permeability hole 114 is not limited to FIGS. 1 and 2 in other embodiments, and the relative position of the air permeability holes 114 from the convex portions 1110 is not limited to that shown in FIG. 1.

In summary, the energy patch of the invention uses the body able to be repeatedly attached to a skin, and the dressing and the energy-transmitting material are disposed in the body. Thus, even after the energy patch has been used many times, the dressing and the energy-transmitting material still can be remained in the body to continuously provide the effect of relieving discomfort of an affected area. Based on the above, the energy patch of the invention has the advantage of being reusable, thereby avoiding material waste. In addition, the body of the energy patch has the convex portions, and the convex portions can simulate the effect of localized muscle massage to more effectively relieve the discomfort of the affected area. On the other hand, because the body further has structures such as the air permeability holes and the concave portions, it can further improve the comfort of the affected area and prevent the energy patch from falling off.

The above are only the preferred embodiments of the present invention and do not limit the present invention in any form, although the present invention has been disclosed as the preferred embodiments, this is not intended to limit the present invention, any person familiar with the art, without departing from the scope of the art of the present invention, the methods and technical contents disclosed above can be used to make slight changes or modifications to equivalent embodiments with equivalent changes, any content that does not deviate from the art of the present invention, any simple modifications, equivalent changes and modifications made to the above embodiment based on the technical essence of the present invention still fall within the scope of the technical solution of the present invention.

What is claimed is:

1. An energy patch, characterized in that the energy patch comprises:
a body, adapted to be repeatedly attached to a skin of human body;
a dressing, disposed in the body; and
an energy-transmitting material, disposed in the body and the energy-transmitting material adapted to transmit energy generated by the dressing.

2. The energy patch according to claim 1, characterized in that, a material of the body comprises silicone, rubber, plastic, polyester, artificial fiber, acrylic, rayon, acetate, elastane, lycra, nylon, latex, polyether polyurethane, foam, liquid gel, glass, stainless steel, steel, or iron.

3. The energy patch according to claim 1, characterized in that, the energy-transmitting material comprises graphene.

4. The energy patch according to claim 1, characterized in that, the dressing comprises saussurea involucrata and a far-infrared radiation material.

5. The energy patch according to claim 1, characterized in that, the dressing and the energy-transmitting material are in a powdered form and are evenly distributed in the body.

6. The energy patch according to claim 5, characterized in that, the body has a surface and an attachment surface opposite to the surface, and the surface has a plurality of convex portions.

7. The energy patch according to claim 1, characterized in that, the body has a plurality of air permeability holes.

8. The energy patch according to claim 7, characterized in that, hole diameters of the air permeability holes are between 0.1 and 1 mm.

9. The energy patch according to claim 7, characterized in that, the air permeability holes are distributed evenly.

10. The energy patch according to claim 1, characterized in that, the body has an attachment surface and two side edges, the two side edges are respectively located on two opposite sides of the attachment surface, and the two side edges each have a concave portion recessed toward each other.
